(19) United States
(12) Patent Application Publication
(10) Pub. No.: US 2010/0041897 A1
(43) Pub. Date: Feb. 18, 2010

(54) Process for preparing a crystalline form of candesartan cilexetil

(75) Inventors: Mercedes Vicioso Sanchez, Madrid (ES); Natividad Rodriguez Salgado, Tres Cantos (Madrid) (ES); Antonio Cosme Gomez, Torres de Alameda (Madrid) (ES); Francisco Eugenio Palomo Nicolau, Alcala de Henares (Madrid) (ES); Pedro Gonzalez Hernandez, Alcala de Henares (Madrid) (ES)

Correspondence Address: PROPAT, L.L.C., 425-C SOUTH SHARON AMITY ROAD, CHARLOTTE, NC, 28211-2841, US

(73) Assignee: CHEMO IBERICA, S.A., Barcelona (ES)

(21) Appl. No.: 12/515,301

(22) PCT Filed: Nov. 22, 2007
(86) PCT No.: PCT/EP2007/062711

§ 371 Date: May 26, 2009

(30) Foreign Application Priority Data
Nov. 23, 2006 (ES) P 200603030

(51) Int. Cl. C07D 403/10

(52) U.S. Cl. 548/250

(57) ABSTRACT

A process for preparing a crystalline form of candesartan cilexetil referred to as Form I, which has a content of Form II lower than 1% (w/w), is described. The process includes a seeding stage with a crystalline product, which mainly includes Form I, and a digestion stage. The invention also relates to the crystalline form, the pharmaceutical compositions containing it and its use for the preparation of a medicament for the treatment of hypertension. Candesartan cilexetil Form I has shown good stability under accelerated stability conditions, when the content of Form II is lower than 1% w/w.

### FIELD OF THE INVENTION

The present invention relates to a crystalline form of candesartan cilexetil, to a process for preparing this form, to compositions containing it, and to its use in treating hypertension.

### BACKGROUND OF THE INVENTION

Candesartan cilexetil is the INN (International Non-proprietary Name) of 2-ethoxy-1-[[2′-(1H-tetrazol-5-yl)[1,1′-biphenyl]-4-yl]methyl]-1H-benzimidazole-7-carboxylic acid, 1-[[(cyclohexyloxy)carbonyl]oxy]ethyl ester having the following structural formula:

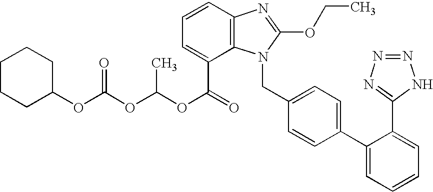

Candesartan cilexetil is an active ingredient used as antihypertensive agent, which was disclosed for the first time in European Patent Application EP-A-0459136.

In Experimental Example 1 of said patent application, a crystalline form referred as C-type is identified by data from the X-ray diffractogram and IR spectrum absorption frequencies.

It is also described that the C-type crystalline form may be obtained by stirring the crude product, or an amorphous product, and/or a crystalline product other than C-type crystalline form, in a suitable solvent at a temperature comprised from −5° C. to 40° C., preferably from 0° C. to 25° C. Among suitable solvents, short-chain alcohols (for example, methanol, ethanol, isopropanol), a mixture of a short-chain alcohol and water, and a mixture of a short-chain ketone (for example, acetone) and water are reported. The solvent:water ratio is preferably comprised from 4:1 to 1:1. The aforesaid patent application also teaches that the amount of solvent is not the limiting but, generally, it is comprised from 2 to 30 times the weight of the product to be crystallized. It is also reported that in the case that a C-type crystalline form is not obtained, C-type crystals may be seeded in order to facilitate crystallization.

Apart from the above general indications about the process for the preparation of the C-type crystalline form, said patent application does not provide any specific embodiment for obtaining such a crystalline form.

Two crystalline forms, Form I and Form II, are described In H. Matsunaga et al. Chem. Pharm. Bull., 1999, 47(2), 182-186, by DSC thermograms, X-ray diffractograms, IR spectra, and solid-state ¹³C-NMR spectra.

Comparison between data of the C form described in EP-A-0459136 and data of Form I described in said article allows concluding that they are the same crystalline polymorph.

Matsunaga et al also describe that Form I is the stable candesartan cilexetil form, and that it may be obtained from the amorphous form or from Form II by recrystallization of any of said compounds in a mixture of acetone and water (3:1 v/v). Said article reports that Form I used in the disclosed assays has a crystallographic purity of 99.4%, but not a specific process for obtaining it is disclosed.

In Comparative Example 2 of the present specification, the crystallization of candesartan cilexetil has been reproduced in acetone/water mixture at a ratio of 3:1 (v/v) following the indications of the abovementioned article, and has been observed that the candesartan cilexetil Form I crystals obtained show a deficient stability in an accelerated stability test performed at 80° C., and possess a high content of Form II as an impurity, which shows itself a high instability in said accelerated stability test.

Therefore, there is a need to provide a process for preparing candesartan cilexetil Form I with a suitable crystallographic purity to achieve a better stability of the active ingredient.

The present inventors have developed a process for the preparation of crystalline candesartan cilexetil Form I with a content of Form II lower than 1% w/w, then showing an improved stability and being suitable for the use in pharmaceutical compositions.

### SUMMARY OF THE INVENTION

An object of the present invention is a process for the preparation of candesartan cilexetil Form I which has a content of Form II lower than 1% (w/w).

The object of the invention further encompasses candesartan cilexetil Form I, wherein the content of Form II is lower than 1% (w/w).

The object of the invention further encompasses candesartan cilexetil Form I obtainable by the process of the invention.

Another object of the invention is a pharmaceutical composition comprising candesartan cilexetil Form I, wherein the content of Form II is lower than 1% (w/w).

The object of the invention further encompasses the use of candesartan cilexetil Form I, wherein the content of Form II is lower than 1% (w/w), for the preparation of a medicament for treating hypertension.

### BRIEF DESCRIPTION OF THE FIGURES

FIG. 1 illustrates the powder X-ray diffractogram for candesartan cilexetil Form I prepared according to the invention. The ordinate represents the number of counts and the abscissa represents the peak position as 2θ angles.

FIG. 2 illustrates the powder X-ray diffractogram for candesartan cilexetil Form II prepared according to Preparative Example 1 included in the present specification. The ordinate represents the number of counts and the abscissa represents the peak position as 2θ angles.

FIG. 3 illustrates the calibration curve for the quantification of the content of candesartan cilexetil Form II in mixtures with Form I. Said calibration curve was constructed from intensities (areas) expressed as counts×°2θ corresponding to a peak at a 2θ angle comprised from 7° to 7.5° of powder X-ray diffractograms recorded on mixtures of candesartan cilexetil Form I and Form II. The ordinate represents the percentage in Form II and the abscissa represents the intensity (area) expressed as counts×°2θ.

FIG. 4 illustrates an enlargement of the zone of the calibration curve comprised from the origin to the value of 2 of respective ordinate and abscissa. The calibration curve is identified by letter A. The two curves identified by letter B at both sides of the calibration curve correspond to the 99% confidence interval, and the curves designated by letter C, which are also situated at both sides of calibration curve and more distant from it than curves B, correspond to 99% prediction limits. The horizontal dotted lines plotted from the ordinate corresponding to 1% and 0.5% of candesartan cilexetil Form I, which is present in Form I, intersect the curve of the prediction limits at the abscissa values corresponding to 23 and 18.5 counts×°2θ, respectively.

### DESCRIPTION OF THE INVENTION

The object of the invention is a process for the preparation of candesartan cilexetil Form I, which has a content of Form II lower than 1% (w/w), comprising:
a) dissolving candesartan cilexetil in a solvent selected from the group consisting of a C₁-C₄ alcohol, a mixture of a C₁-C₄alcohol and water, and a mixture of a C₃-C₄ ketone and water wherein the organic solvent:water ratio is comprised from 4:1 to 1:1 (v/v), and
b) crystallizing the product from said solvent by cooling,
   characterized in that the mixture comprising the product and the solvent:
c) is seeded at a temperature comprised from 40° C. to 70° C. with crystals mainly containing candesartan cilexetil Form I, and
d) is maintained at a temperature comprised from 40° C. to 70° C. for a period comprised from 30 to 90 minutes before cooling.

The candesartan cilexetil used in the process of the invention may be obtained, for example, according to the process described in Example 44 of European Patent application EP-A-0459136. In said example, the preparation of an amorphous product from the purification of the reaction crude by column chromatography and subsequent solvent evaporation is described. Likewise, the preparation of a crystalline product by crystallization of the amorphous product from ethanol is described.

The candesartan cilexetil used as starting material in the process of the invention may be amorphous or crystalline. The crystalline product may comprise only one polymorph or a mixture of polymorphs, for example a mixture of Form I and Form II.

In the process of the invention, candesartan cilexetil is dissolved in a solvent selected from the group consisting of a C₁-C₄ alcohol, a mixture of a C₁-C₄ alcohol and water, and a mixture of a C₃-C₄ ketone and water.

Throughout the present specification the term “solvent” refers to either only one solvent or a solvent mixture, in the case that more than one solvent is included.

Preferably, the solvent used is a mixture of a C₃-C₄ ketone and water, more preferably a mixture of acetone and water.

When an organic solvent/water mixture is used, the organic solvent:water ratio is preferably comprised from 3.5:1 to 2:1 (v/v), more preferably from 3.2:1 to 2.8:1 (v/v).

In a preferred embodiment, candesartan cilexetil is dissolved in acetone under reflux conditions and water is slowly added maintaining a temperature comprised from 55° C. to 57° C. Dissolution of product usually occurs after adding an amount of water corresponding to 10%-50% of the total amount of water to be added.

The amount of solvent used in the process of the invention is usually comprised from 2 to 30 times the weight of the product to be crystallized, preferably from 4 to 10 times.

The process of the invention is characterized in that the mixture comprising the product and the solvent is seeded at a temperature comprised from 40° C. to 70° C., preferably at a temperature comprised from 50° C. to 60° C., and more preferably from 54° C. to 58° C.

For the person skilled in the art, it is a matter of routine to determine the suitable temperature for the solvent system selected within the temperature ranges of the invention, since only the boiling point of the solvent or solvents used should be taken into account.

In the process of the invention, the mixture of the product and the solvent is seeded with crystals mainly comprising candesartan cilexetil Form I. Preferably, the content of Form I in said crystals is higher than 75%, more preferably higher than 90%, even more preferably higher than 95%, and most preferably higher than 99%.

In order to perform the seeding process for the first time, the candesartan cilexetil obtained by the process described in Preparative Example 2 (see below) may be used. Subsequently, the candesartan cilexetil Form I obtained by the process of the invention may be used, which has a content of Form II lower than 1% (w/w), usually even lower than 0.5% (w/w).

Generally, crystals are seeded until a permanent turbidity is achieved in the reaction mass.

Then, after seeding, the product/solvent mixture is maintained at a temperature comprised from 40° C. to 70° C. for a period comprised from 30 to 90 minutes, preferably from 45 to 75 minutes, more preferably from 55 to 65 minutes. Preferably the temperature is maintained from 50° C. to 60° C., and more preferably from 54° C. to 58° C.

Thereafter, the mixture is cooled.

Generally, the mixture is cooled to room temperature, from 20° C. to 30° C., which produces an abundant precipitation. Preferably, the cooling process is continued until it reaches a temperature comprised from 0° C. and 10° C., this temperature is maintained for a period of about 1 hour. This period of time may be longer so as to ensure a complete precipitation.

Once precipitation is considered as complete, the crystalline product is recovered, for example, by filtration, and it is generally dried under vacuum in a stove at a temperature comprised from 30° C. to 50° C.

By the process of the invention, candesartan cilexetil Form I is obtained in good yield, generally comprised from 80% to 95% with respect to the starting material.

The process of the invention allows to obtain candesartan cilexetil Form I with a content of Form II lower than 1% (w/w), generally lower than 0.5% (w/w), and even lower than 0.1% (w/w).

The crystallographic purity of the obtained product may be quantitatively determined by powder X-ray diffraction using a calibration curve which has been constructed as hereinafter explained.

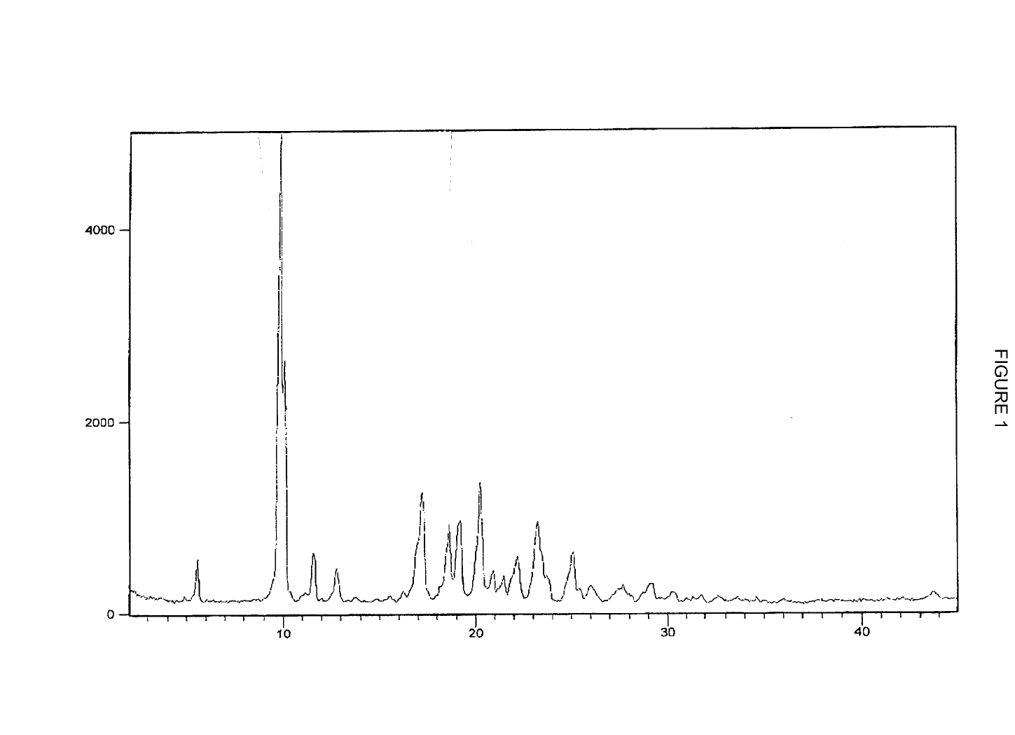

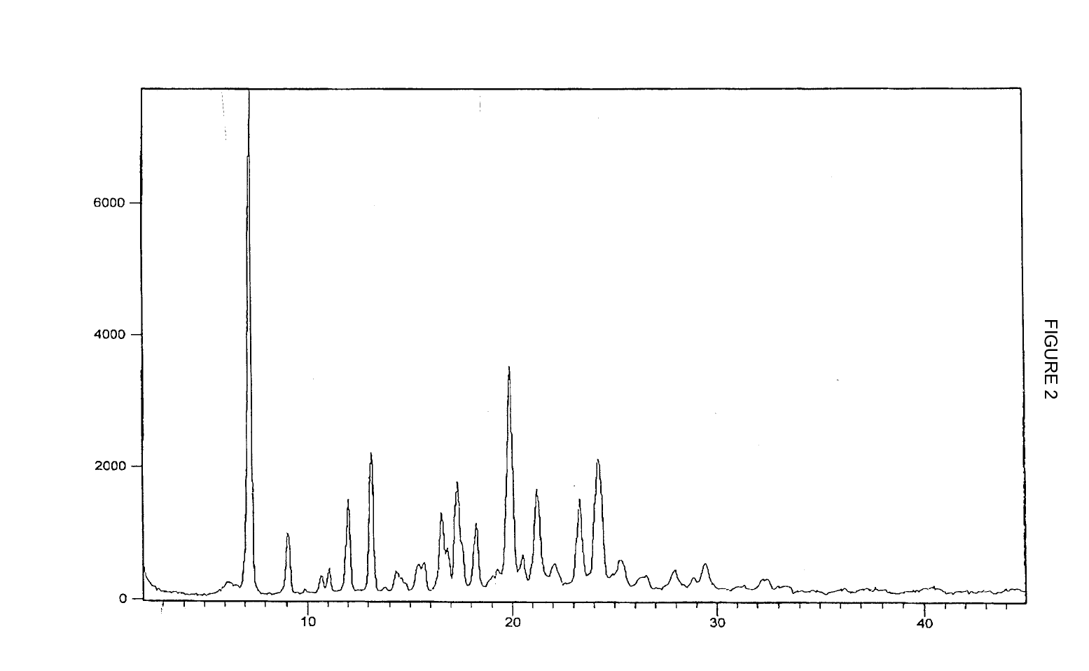

Firstly, the powder X-ray diffractograms of candesartan cilexetil Form I (FIG. 1), prepared according to the process of the invention (Example 2), and candesartan cilexetil Form II (FIG. 2), prepared according to Preparative Example 1, were recorded.

Then the powder X-ray diffractograms and their peak list were compared, and a characteristic peak of Form II at a 2θ angle comprised from 7° to 7.5° which significantly differed it from Form I, was identified. The peak was exactly situated at 7.26° for Cu K_{α} radiation, which corresponds to a spacing of 12.15 Å.

This estimate is coincidental with that in the abovementioned study by Matsunaga et al, wherein is disclosed that Form II have a characteristic peak at a 2θ angle of 7.28°, which is the peak exhibiting the highest intensity in the powder X-ray diffractogram for Form II.

Then standard samples were prepared containing candesartan cilexetil Form I, obtained according to Example 2, and 0.5%, 1%, 2%, 5%, 10% and 20% w/w of candesartan cilexetil Form II, obtained according to Preparative Example 1.

The samples were grinded in an agate mortar and placed in the rotary sample holder of the X-ray diffractometer, and the powder X-ray diffractogram of each sample was recorded.

The powder X-ray diffractograms were recorded in a PANALYTICAL XPERT-PRO instrument with XCELERATOR detector fitted with a copper tube, graphite monochromator and automatic slot; the tube was operated at a voltage of 40 kV and an intensity of 40 mA. The rotary sample holder was scanned continuously in the 2θ range between 2° and 45°, with a step size of 0.05°. The irradiated length was 12 mm

Computer programs associated to X-ray diffraction allow obtaining listings wherein, among other data, 2θ angle peaks and also the intensities expressed as areas corresponding to each peak are shown. The units of the intensity expressed as area are counts×°2θ, wherein the counts are measured on the angle range of the peak. Table I shows the values of said intensity for the peak at a 2θ angle comprised from 7° to 7.5° corresponding to candesartan cilexetil Form I samples containing different percentages by weight of Form II:

TABLE I

| Area (counts × °2θ) | % wt of Form II |
|---|---|
| 5.47 | 0.5 |
| 12.67 | 1 |
| 19.13 | 2 |
| 42.53 | 5 |
| 81.52 | 10 |
| 140.63 | 20 |

From these data, a calibration curve using polynomial adjustment was constructed, which responds to the equation:

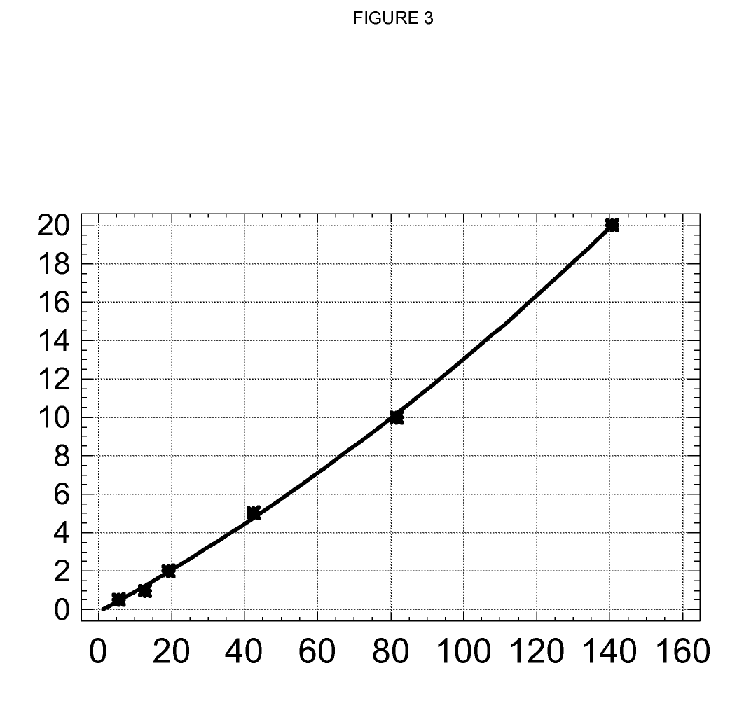

Y=0.0003X²+0.1034X−0.1357

wherein Y is % of Form II by weight, and X is the intensity expressed as peak area at a 2θ angle comprised from 7° to 7.5°. The regression coefficient of the calibration curve is (R²) of 0.9995, and it is shown in FIG. 3.

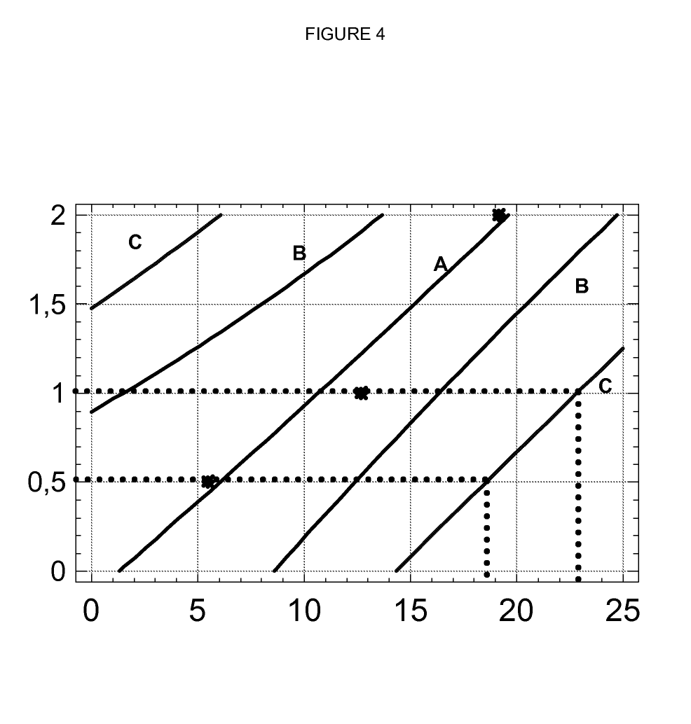

For the purpose of the present invention and taking into account the calibration curve and the 99% prediction limits (see FIG. 4), candesartan cilexetil Form I is considered to have a content of Form II lower than 1% w/w, if the intensity expressed as peak area at a 2θ angle situated between 7° and 7.5° has a value lower than 23 counts×°2θ. Analogously, candesartan cilexetil Form I is considered to have a content of Form II lower than 0.5% w/w if the intensity expressed as peak area at a 2θ angle situated between 7° and 7.5° has a value lower than 18.5 counts×°2θ.

It can be seen that the calibration curve in FIG. 3 approximately goes through the origin (point (0.0), that can be interpreted as the candesartan cilexetil Form I prepared according to the process of the invention, as in Example 2, is substantially free of Form II, i.e. the content of Form II can be considered to be even lower than 0.1% w/w.

The object of the invention encompasses candesartan cilexetil Form I, wherein the content of Form II is lower than 1% w/w, preferably lower than 0.5%, and more preferably lower than 0.1% w/w.

The invention also encompasses candesartan cilexetil Form I which is obtainable by the process of the invention.

The authors have performed several crystallization assays in accordance with prior art disclosure and have obtained candesartan cilexetil Form I unpurified with other crystalline forms, particularly Form II, which shows a poor stability when it is maintained at 80° C. under accelerated stability testing conditions for a period comprised from 5 to 9 days.

Table II summarizes the results of said crystallization assays of candesartan cilexetil using an acetone/water mixture (3:1 v/v) as a solvent as reported in the abovementioned study by Matsunaga et al.

TABLE II

| Assay | Seed | Digestion | Result | Stability |
|---|---|---|---|---|
| 1 | No | No | Mixture of crystalline forms | Poor |
| 2 | Yes | No | Mixture of crystalline forms | Poor |
| 3 | No | Yes | Mixture of crystalline forms | Poor |
| 4 | Yes | Yes | Form I with less than 1% of | Good |
| | | | Form II | |

Seeding was carried out with a crystalline product mainly comprising Form I, and the digestion was carried out at a temperature ranging from 40° C. to 70° C. for a time period comprised from 30 to 90 minutes.

It can be seen that Assays 1, 2 and 3 lead to candesartan cilexetil which is a mixture of crystalline forms, while Assay 4, which corresponds to the process of the invention, leads to candesartan cilexetil Form I with a content of Form II lower than 1% w/w.

Surprisingly, the present inventors have discovered that on applying a digestion stage after seeding with a crystalline product mainly comprising Form I, candesartan cilexetil Form I is obtained substantially free of the polymorph designed as Form II, and it shows a high stability when it is maintained under the conditions of a performed accelerated stability testing.

In order to determine the influence of Form II on the stability of candesartan cilexetil Form I, samples with different proportions of Form II were prepared and subjected to an accelerated stability test at a temperature of 80° C. for different periods of time up to 9 days. Once the time period elapsed, the samples were analyzed by HPLC for the determination of the content of impurities, according to the method described in the Examples.

The results obtained in the above test revealed the elevated instability shown by candesartan cilexetil Form II itself, since after remaining at 80° C. for 5 days, it shown a content of impurities of 60% (w/w), calculated from the areas of the HPLC chromatogram.

On the other hand, candesartan cilexetil Form I prepared according to the process of the invention had a content of impurities of 2% w/w, even below 0.5% w/w after remaining at 80° C. for 7 days. The content of impurities after the accelerated testing study increased as the proportion of Form II in the mixture increased, as shown in the assays described in Example 3.

The invention also encompasses the compositions comprising candesartan cilexetil Form I obtainable by the process according to this invention and/or having a content of Form II lower than 1% w/w, preferably lower than 0.5% w/w, and even more preferably lower than 0.1% w/w.

The pharmaceutical compositions containing the product of the invention may include pharmaceutically acceptable excipients, adjuvants, vehicles, and/or diluents, for the purposes of the formulation of presentation forms according to conventional methods well known to those skilled in the art. Presentation forms include powders, granules, tablets, capsules, injections, syrups, emulsions, elixirs, suspensions, solutions, suppositories. The route of administration may be oral, parenteral, inhalation, rectal, or topical.

The invention also encompasses the use of candesartan cilexetil Form I obtainable according to the process of the invention and/or having a content of Form II lower than 1% w/w, for the preparation of a medicament for treating hypertension, preferably lower than 0.5% w/w, and even more preferably lower than 0.1% w/w.

The following examples are given in order to provide those skilled in the art with a detailed description of a specific embodiment of the process for preparing the compound of the invention.

### EXAMPLES

### Preparative Example 1

### Preparation of Candesartan Cilexetil Form II

10.5 g of crude candesartan cilexetil, obtained, for example, according to the process described in the patent application EP-A-0459136 were weighed, dissolved in 63 ml (6 volumes) of acetone and heated under reflux at a temperature of 55° C.

The resulting solution was gradually cooled at room temperature (20° C.-30° C.) until a precipitate appeared. In the case that a precipitate does not appear, the solution may be concentrated by removing half of the solvent.

Then the suspension was stirred with the precipitate at a temperature ranging from 0° C. to 10° C. for 1 hour.

The product was separated by filtration, dried under vacuum in a stove at 40° C. 10.1 g of pure candesartan cilexetil Form II were obtained (96.7% yield).

The crystalline form of the resultant product was characterized by the recording of the powder X-ray diffractogram (FIG. 2), which is substantially coincident with the powder X-ray diffractogram and the listing of peaks at 2θ angles provided in the abovementioned article by Matsunaga et al.

### Preparative Example 2

### Preparation of Seed Crystals of Candesartan Cilexetil Form I

32.8 kg of crude candesartan cilexetil obtained, for example, according to the process described in the patent application EP-A-0459136 were weighed. 83 kg (105.3 l) of acetone, and 16 kg of purified water were added and the mixture was heated under reflux. Reflux temperature reached 55° C. after 1 hour.

Then, 5.2 kg of purified water were added, and heating at the temperature of 55° C. was maintained until complete dissolution of the product which took place within about 15 minutes.

The resulting solution was filtered using 1 kg of active charcoal and 2 kg of an auxiliary filtration agent for removal of incidental coloration.

The filtered solution was maintained at a temperature of 40° C. and 28 kg (35.5 l) of acetone and 26 kg of water were added.

The acetone:water ratio used in the crystallization was 3:1 (v/v).

The solution was heated to 55° C. in 30 minutes and became turbid. Once the temperature of 55° C. has been reached, the reaction mass was maintained at this temperature for 1 hour.

Then it was cooled until a temperature of 24° C. in 2 hours. A large amount of precipitate appeared, and cooling was continued until −2° C. in 5 hours.

The suspension was centrifuged to separate the solid product, which was dried in a stove at 40° C. under vacuum. 28.4 kg (yield 87%) of candesartan cilexetil Form I containing 19% (w/w) of Form II were obtained.

The content of Form II was quantitatively determined by X-ray diffraction on the basis of the aforesaid calibration curve.

### Example 1

### Preparation of the Candesartan Cilexetil Form I of the Invention

10 g crude candesartan cilexetil obtained, for example, according to the process described in the patent application EP-A-0459136 were weighed, dissolved in 41 ml (4.1 volumes) of acetone, and the mixture was heated under reflux at a temperature of 55° C.-57° C. Then 13.25 ml (1.32 volumes) of water were gradually added and the temperature was maintained at about 55° C. After the addition of 10-15% of the total water, the complete dissolution of the product was observed.

The acetone:water ratio used was 3.1:1 (v/v)

Once water addition has ended, it was seeded with crystals of candesartan cilexetil obtained in Preparative Example 2 until a permanent turbidity was obtained.

The mixture was stirred at 55° C. for 1 hour, and then gradually it was cooled to room temperature (20° C.-30° C.) in about 1 hour under further stirring. The apparition of a large amount of precipitate was observed.

Then the suspension was cooled to a temperature ranging from 0° C. to 10° C. in an ice-methanol bath in about 30 minutes, and it was stirred at this temperature for 1 hour.

The precipitate was separated by filtration and washed with 9.5 ml of an acetone/water mixture (3:1 v/v).

The resulting product was dried in a stove at 40° C. under vacuum and 9.1 g (yield 91%) of candesartan cilexetil Form I were obtained with a content of Form II lower than 0.5% w/w. This value was determined using the abovementioned calibration curve.

### Example 2

### Preparation of Candesartan Cilexetil Form I of the Invention

20 g crude candesartan cilexetil obtained, for example, according to the process described in the patent application EP-A-0459136 were weighed, dissolved in 60 ml (3 volumes) of acetone, and the mixture was heated under reflux at a temperature of 55° C.-57° C. Then 20 ml (1 volume) of water were gradually added and the temperature was maintained at about 55° C.

Once water addition has ended, seeding with crystals of candesartan cilexetil Form I, as obtained in Preparative Example 1, which has a content of Form II lower than 0.5% (w/w), was carried out.

The mixture was stirred at 55° C. for 1 hour and then gradually cooled at room temperature (20° C.-30° C.) under stirring until a large amount of precipitate was produced.

Then 19 ml of an acetone/water mixture (3:1 v/v) were added, and the suspension was cooled to a temperature comprised from 0° C. to 10° C., and maintained at said temperature for 1 hour under stirring.

The precipitate was separated by filtration and washed with 19 ml of an acetone/water mixture (3:1 v/v).

The resulting product was dried at 40° C. under vacuum and 18.4 g of candesartan cilexetil Form I substantially free of Form II were obtained (92% yield).

The candesartan cilexetil Form I obtained in this Example was used for the preparation of samples containing Form II as an impurity, which were employed in the recording of the powder X-ray diffractogram to construct the calibration curve for the quantitative determination of the content of Form II in samples containing it.

### Example 3

### Accelerated Stability Test

Samples of candesartan cilexetil Form I containing different proportions of Form II were prepared from the products obtained in Example 2 and Preparative Example 1. The samples were exposed to an accelerated stability test at a temperature of 80° C. for different time periods up to 9 days.

Once the time period has elapsed, the samples were analyzed by HPLC for the determination of the content of impurities.

Impurities corresponding to the following retention times: 8.3, 10.4, 12.5, 14.1 and 16.4 minutes were identified in the HPLC chromatograms.

The HPLC analysis of impurities was carried out in a HPLC system with UV detector (Waters Alliance), with a L1-type column according to US Pharmacopeia nomenclature, at a temperature of 30° C., with a flow rate of 1 ml/min, an injection volume of 10 μl, and an UV detector at 210 nm.

The mobile phase was constituted by a mixture of solution A (v/v) (aqueous 0.1% v/v trifluoroacetic acid buffer) and acetonitrile.

During the analysis the mobile phase contained 45% of Solution A and 55% of acetonitrile, except for the period comprised from minute 15 to minute 25 wherein a mixture of 5% Solution A and 95% acetonitrile was used.

The sample was prepared dissolving 0.2 mg of the sample in 1 ml of mobile phase.

TABLE III shows the results of the accelerated stability test performed at a temperature of 80° C. for the shown period, corresponding to the products prepared in Example 2 (Form I), in Preparative Example 1 (Form II), and mixtures prepared with said products:

TABLE III

| | | Sum of impurities |
|---|---|---|
| Sample | Time (days) | (% HPLC area) |
| Form I (Example 2) | 7 | 0.34 |
| Form II (Preparative Example 1) | 5 | 59.98 |
| Form I + 0.1% of Form II | 9 | 0.96 |
| Form I + 0.3% of Form II | 9 | 1.21 |
| Form I + 0.5% of Form II | 7 | 1.46 |
| Form I + 1.0% of Form II | 7 | 2.00 |
| Form I + 2.0% of Form II | 7 | 2.88 |
| Form I + 5.0% of Form II | 7 | 6.65 |
| Form I + 10.0% of Form II | 7 | 12.78 |
| Form I + 20.0% of Form II | 7 | 23.20 |

The sum of the impurities refers to the sum of areas recorded in the HPLC chromatogram corresponding to the degradation impurities detected at the retention times above specified.

It can be observed that the candesartan cilexetil Form I substantially free of Form II prepared according to the process of the invention shows a good stability, while Form II is itself unstable and generates a considerable amount of impurities when tested under conditions of accelerated stability.

When increasing the proportion of candesartan cilexetil Form II in the samples, it can be observed that the percentage of impurities produced in the accelerated stability test also increases.

1. A process for preparing candesartan cilexetil Form I, which has a content of Form II lower than 1% w/w, comprising:
a) dissolving candesartan cilexetil in a solvent selected from the group consisting of a C₁-C₄ alcohol, a mixture of a C₁-C₄ alcohol and water, and a mixture of a C₃-C₄ ketone and water wherein the organic solvent:water ratio is comprised from 4:1 to 1:1 (v/v), and
b) crystallizing the product from said solvent by cooling,
wherein the mixture comprising the product and the solvent:
c) is seeded at a temperature of from 40° C. to 70° C. with crystals mainly comprising candesartan cilexetil Form I, and
d) is maintained at a temperature of from 40° C. to 70° C. for a period of from 30 to 90 minutes before cooling.

2. A process according to claim 1, wherein the solvent used is a mixture of a C₃-C₄ ketone and water.

3. A process according to claim 2, wherein the solvent is a mixture of acetone and water.

4. A process according to claim 1 wherein the organic solvent:water ratio is from 3.5:1 to 2:1 (v/v).

5. A process according to claim 4, wherein the organic solvent:water ratio is from 3.2:1 to 2.8:1 (v/v).

6. A process according to claim 1, wherein the mixture comprising the product and the solvent is seeded at a temperature of from 50° C. to 60° C.

7. A process according to claim 6, wherein the seeding is carried out at a temperature of from 54° C. to 58° C.

8. A process according to claim 1, wherein the content of Form I in seed crystals is higher than 75%.

9. A process according to claim 8 wherein the content of Form I in seed crystals is higher than 90%.

10. A process according to claim 9, wherein the content of Form I in seed crystals is higher than 95%.

11. A process according to claim 10, wherein the content of Form I in seed crystals is higher than 99%.

12. A process according to claim 1, wherein the time period of step d) is from 45 to 75 minutes.

13. A process according to claim 12, wherein the time period of step d) is from 55 to 65 minutes.

14. A process according to claim 1, wherein the temperature of step d) is from 50° C. to 60° C.

15. A process according to claim 14, wherein the temperature of step d) is from 54° C. to 58° C.

16. (canceled)

17. (canceled)

18. (canceled)

19. (canceled)

20. (canceled)

21. (canceled)

22. A process according to claim 3, wherein the organic solvent:water ratio is from 3.5:1 to 2:1 (v/v).

23. A process according to claim 22, wherein the mixture comprising the product and the solvent is seeded at a temperature of from 54° C. to 58° C.

24. A process according to claim 23, wherein the content of Form I in seed crystals is higher than 95%.

25. A process according to claim 24, wherein the time period of step d) is from 55 to 60 minutes.

26. A process according to claim 25, wherein the temperature of step d) is from 54° C. to 58° C.
